(19) United States
(12) Patent Application Publication
(10) Pub. No.: US 2022/0160479 A1
(43) Pub. Date: May 26, 2022

(54) SYSTEMS AND PROCESSES OF CREATING DIGITAL DESIGNS OF DENTAL APPLIANCES

(71) Applicant: Dean UltraThin Retainer, LLC, Fosston, MN (US)

(72) Inventors: Bryce Oakes, Fosston, MN (US); Austin Peterson, Fosston, MN (US)

(21) Appl. No.: 17/456,184

(22) Filed: Nov. 23, 2021

Related U.S. Application Data
(60) Provisional application No. 63/117,132, filed on Nov. 23, 2020.

(51) Int. Cl. A61C 13/00; A61C 7/10; A61C 7/08; A61C 5/85; B33Y 50/00; B22F 10/80

(52) U.S. Cl. CPC A61C 13/0019; A61C 7/10; A61C 7/08; B33Y 30/00; B33Y 50/00; B22F 10/80; A61C 5/85

(57) ABSTRACT

Processes and systems for creating digital designs of patient specific dental appliances and parts thereof, such as dental bands and wires. After receiving an order for a dental appliance that includes data regarding the patient's teeth and optionally adjacent anatomy, the digital design of the dental appliance is automatically started by dental appliance design software using rules based logic. Thereafter, a human can continue, and in some embodiments complete, the digital design. Or the entire design can be completed by the dental appliance design software without any human intervention. The final digital design can then be used to create the patient specific dental appliance.

### FIELD

This technical disclosure relates to manufacturing dental appliances and to creating digital designs of dental appliances which are designed specifically to match anatomy of patients intended to wear the dental appliances.

### BACKGROUND

Dental appliances (also known as orthodontic appliances) have been in use for years in the dentistry field for providing a variety of benefits, for example retention after completion of an active phase of orthodontic treatment. In some instances, dental appliances and/or parts thereof are manufactured in a “generic” manner whereby the dental appliances and/or parts thereof do not match the specific anatomy of the patient.

### SUMMARY

Processes and systems for creating digital designs of patient specific dental appliances and parts thereof, such as dental bands and wires, are described. After receiving an order for a dental appliance, where the order includes data regarding the patient's teeth and optionally adjacent anatomy, the digital design of the dental appliance is automatically started, for example using a rules-based logic system, preferably without initial human intervention. In some embodiments, the digital design may be automatically completed. In other embodiments, a human can continue, and in some embodiments complete, the digital design. The final digital design can then be used to create the patient specific dental appliance. Automatically starting the digital design can include, but is not limited to, using the data of the patient's teeth to automatically begin the design of the dental appliance such as designing some or all of the design of a dental band, a wire of the dental appliance, or any other component of the dental appliance using programmed software that employs rules-based logic.

The digital design of the dental appliance can be performed using dental appliance design software. The design software can be controlled by or incorporate artificial intelligence software which can automatically start the digital design using rules-based logic programming once the order is received. The start of the digital design can result in creation of a partial digital design. In some embodiments, the entire digital design can be completed using the dental appliance design software. In some embodiments, a human can be involved in the design process, for example reviewing the partial or complete digital design for accuracy, altering the digital design, continuing the creation of the digital design based on the partially completed design, and optionally completing the digital design.

The term “dental appliance” is used herein interchangeably with “orthodontic appliance” and is intended to encompass any device used in dental/orthodontic treatment. Non-limiting examples of dental appliances that can be digitally designed as described herein include, but are not limited to, space maintainers, palatal expanders, hyrax appliances, distalizing appliances, retainers, habit appliances, partial frameworks, and other dental appliances. A dental appliance also includes dental bands, wires, clasps and individual components of a dental appliance.

The final or completed digital design can be provided to a suitable manufacturing machine for use in creating the patient specific dental appliance. In one embodiment, the dental appliance can be created using a suitable additive manufacturing process. Additive manufacturing as used herein is intended to encompass any process where the dental appliance is created by adding layer-upon-layer of material to create the dental appliance. Additive manufacturing may also be referred to as 3D printing. In another embodiment, the dental appliances can be created using a subtractive manufacturing process. In additional embodiments, the dental appliances can be created using other manufacturing techniques as well, for example by casting.

The dental appliances described herein can be formed of any material(s) suitable for forming dental appliances including, but not limited to, metals including but not limited to pure metals such as gold, platinum, or titanium or metal alloys such as stainless steel, nickel titanium or a cobalt, chromium, molybdenum alloy; graphene; carbon; carbon fiber; plastic; and ceramic.

In one embodiment, a process of creating a dental appliance can include digitally receiving an order for the dental appliance to be created, where the order includes data on a patient's teeth and an identification of the dental appliance to be created. A portion of a digital design of the dental appliance is then automatically created using data from the order without human interaction based on rules-based programming logic. Thereafter, creation of the digital design of the dental appliance may be continued using human interaction or the digital design can be completed using the rules-based programming logic. The digital design is stored in data storage at any time during the digital design process.

In another embodiment, an additive manufacturing system that produces a dental appliance is described. The system includes an additive manufacturing machine for creating the dental appliance, and a control computer connected to and controlling the additive manufacturing machine. A system is in direct or indirect communication with the control computer or with the additive manufacturing machine, where the system provides a digital design of the dental appliance to the control computer or to the additive manufacturing machine. In some embodiments, the system may be in direct wired or wireless communication with the control computer or with the additive manufacturing machine to directly supply the digital design to the control computer or to the additive manufacturing machine. In another embodiment, the digital design may by stored on a removable storage device, such as on a USB drive, with the digital design then being transferred to the control computer or the additive manufacturing machine via the removable storage device.

### DRAWINGS

FIG. 1 schematically illustrates an example of a process of creating a digital design and a patient specific dental appliance.

FIG. 2 illustrates an example of information in a dental appliance order.

FIG. 3 schematically illustrates an example of a system for creating a patient specific dental appliance from a digital design.

FIG. 4 illustrates an example of the server in the system of FIG. 3.

FIG. 5 illustrates an additive manufacturing system that can be used to create the patient specific dental appliance from the digital design.

FIG. 6 depicts an example of a digital design of a patient specific dental appliance that can be created.

FIG. 7 depicts another example of a digital design of a patient specific dental appliance that can be created.

FIG. 8 depicts another example of a digital design of a patient specific dental appliance that can be created.

### DETAILED DESCRIPTION

The following is a description of creating digital designs of patient specific dental appliances. After receiving an order for a dental appliance, where the order includes digital data of the patient's tooth or teeth and optionally adjacent anatomy, the digital design of the dental appliance is automatically started using programmed software using rules-based programmed logic. In one embodiment, the starting of the digital design can be performed without any human interaction. Thereafter, a human can continue, and in some embodiments complete, the digital design. In one embodiment, the entire digital design of the dental appliance can be created without any human interaction. The final or completed digital design can then be used to create the dental appliance. As used herein, digital data of a patient's teeth include digital data of a single tooth or multiple teeth of the patient.

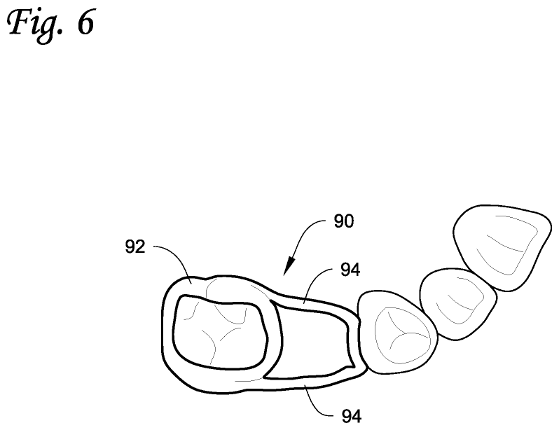

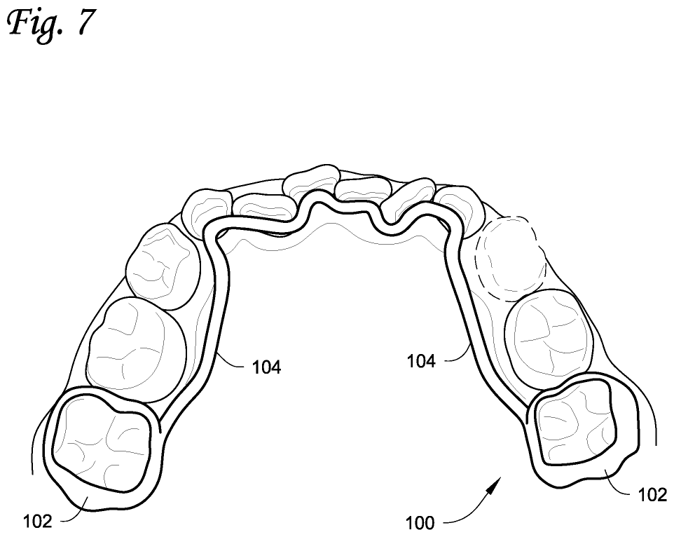

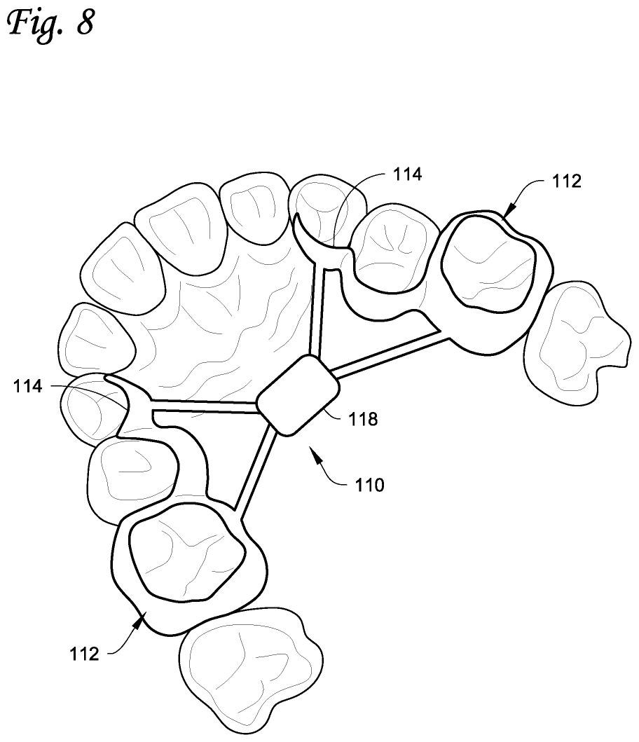

The digital design is based on the specific anatomy of each patient so that the resulting manufactured dental appliance more closely conforms to the patient's anatomy. In some embodiments, the dental appliance may be prescribed for the patient by the patient's dentist/orthodontist or other medical professional. The dental appliances can be any dental appliances that are worn by patients during a course of dental/orthodontic treatment. Non-limiting examples of dental appliances can include, but are not limited to, space maintainers (see FIGS. 6 and 7), palatal expanders (see FIG. 8), distalizing appliances, retainers, habit appliances, dental bands (FIGS. 6-8), wires (FIGS. 6-8), replacement tooth/teeth, and other dental appliances. The dental appliances can be designed for use with a patient's upper set of teeth or lower set of teeth.

The dental appliances can be formed from any material that provides the requisite durability and strength for the dental appliances. In one embodiment, the material can be metal. The metal can be any metal suitable for use in dental applications and that provides the requisite durability and strength to perform the functions of the dental appliances. Examples of metals that can be used include, but are not limited to, gold, platinum, titanium, and metal alloys such as stainless steel, or an alloy primarily composed of cobalt, chromium and molybdenum. In another embodiment, the material can be a non-metallic material including, but not limited to, plastic, graphene, carbon, and carbon fiber.

The digital designs described herein can be used to create the dental appliances using any type of manufacturing process that the digital designs can be used with. In one embodiment, the digital design can be used by an additive manufacturing machine to create the dental appliance using additive manufacturing. Additive manufacturing as used herein is intended to encompass any process where the dental appliance is created by adding layer-upon-layer of material to create the dental appliance. Additive manufacturing may also be referred to as 3D printing. In another embodiment, the dental appliances can be created using a subtractive manufacturing process. In additional embodiments, the dental appliances can be created using other manufacturing techniques as well, for example by casting.

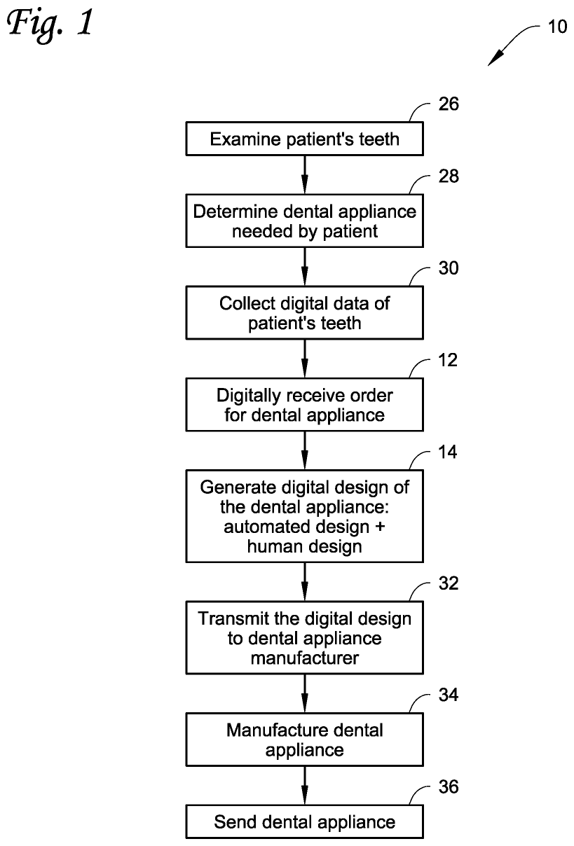

Referring initially to FIG. 1, an example of a process 10 of creating a digital design and a patient specific dental appliance is illustrated. In the process 10, an order for a dental appliance is digitally received at 12. The order may be a prescription from the patient's dentist/orthodontist or other medical professional and be sent by the doctor/medical professional. In another embodiment, the order may be made by the patient, with or without a doctor's prescription. In still another embodiment, the order may come from a dental lab. The order is then used to generate the digital design of the dental appliance at 14.

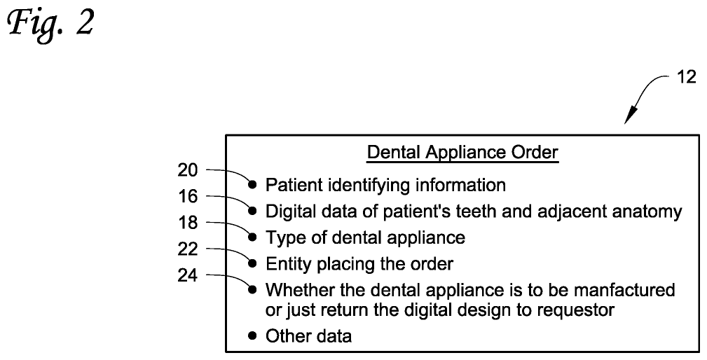

Referring to FIGS. 1 and 2, the order 12 includes sufficient information to be able to create a digital design of the patient specific dental appliance. For example, as illustrated in FIG. 2, the order 12 can include at least digital data of the patient's teeth 16, the type of dental appliance 18 being requested, and patient identifying information 20 such as a patient identification number or the name of the patient. The order 12 may also include data such as the entity placing the order 22, whether the dental appliance is to be manufactured or the digital design is to be returned to the requestor 24, where the dental appliance is to be sent once/if manufactured, and other data. In some embodiments, for example when the digital design software is available on a software as a service model, payment and/or log-in information may also be included in the order 12 or provided separately. In some embodiments, payment information may be included in the order 12.

The type of dental appliance 18 can include specifics on the doctor's prescription for the patient. For example, depending upon the type of dental appliance being requested, the information included with the order 12 can include specifics on one or more of: the clasp design, clasping design, banding design, precision attachment design, palatal coverage, post dam, tooth/teeth to be replaced, occlusion, and the like.

Returning to FIG. 1, data for the order 12 can be initially collected by a dentist/orthodontist examining the patient's teeth at 26 in order to determine a course of treatment and determine the dental appliance needed by the patient at 28. Digital data of the patient's teeth and optional adjacent anatomy is collected at 30. The digital data can be obtained using conventional digital scanning techniques such as performing a scan of an impression of the patient's teeth, using an intra-oral digital scanner to intraorally scan the patient's teeth, or digitally scanning a model of the patient's teeth. Examples of intra-oral scanners include, but are not limited to, Planmeca's PlanScan, 3Shape's TRIOS, Sirona's Apollo DI and OmniScan, Invisalign's Itero and 3M's True Definition Scanner. The digital data can also be generated based on a dental cone beam CT scan. Techniques for obtaining digital data of patient's teeth are well known in the art. The steps 26, 28, 30 can be performed in any order relative to one another. In addition, each one of the steps 26, 28 30 need not be performed if the steps have previously been performed and the data and other information from the previously performed steps is considered sufficiently accurate to place the order 12 for the dental appliance. In another embodiment, some or all of the data for the order 12 can be collected by the patient, for example by the patient scanning their own teeth or scanning an impression of their teeth.

In one embodiment, examination of the patient's teeth 26 may be omitted and determination of the dental appliance 28 can be performed automatically, without examination by a dentist/orthodontist, using software to analyze the collected digital data. For example, the patient may have had a recent examination from which the data necessary to generate the digital design can be obtained. In addition, the collection of the data 30 can be performed by a dentist/orthodontist, by a dental assistant, by a lab technician, or even by the patient.

Generating the digital design of the dental appliance at 14 includes two primary steps. First, after the order 12 is received, the dental appliance design software (described further below) automatically begins creation of the digital design of the requested appliance and automatically creates a portion of the digital design, preferably without any human interaction. Even though the requested dental appliance is specific to the patient, in many instances features of dental appliances are common between dental appliances and are not altered by the patient anatomy. In such instances, the dental appliance design software can automatically create the common features of the digital design. In addition, the dental appliance design software, optionally supplemented by artificial intelligence software, can analyze the patient specific tooth data provided in the order 12 and use the data to generate some or all of the features of the dental appliance that are patient specific. For example, in the case of a dental band, the patient specific tooth data can be automatically analyzed upon receipt and the digital design of the dental band automatically created based on the tooth data without the need for any human interaction.

After the portion of the digital design is automatically created, a human technician may become involved in the digital design. For example, the human technician can review the automatically created portion of the digital design and if necessary revise the automatically created portion. The human technician can continue the digital design, and in some embodiments the human technician can complete the digital design. In other embodiments, the dental appliance design software can finish the digital design after the human technician completes their interaction with the digital design. In other embodiments, the dental appliance design software can generate the entire digital design of the dental appliance without any human interaction.

With continued reference to FIG. 1, once the digital design is finished, the digital design can then be used to manufacture the patient specific dental appliance. In one embodiment, the digital design can be transmitted back to the entity that placed the order or transmitted to another requested location, with the digital design then being used to manufacture the patient specific dental appliance using suitable manufacturing equipment of the requestor or other entity. Alternatively, the digital design can be transmitted to a dental appliance manufacturer at 32 and the patient specific dental appliance is manufactured at 34. The patient specific dental appliance can then be sent at 36, for example to the patient's doctor, to a dental lab hired by the patient's doctor, directly to the patient, or to any other location.

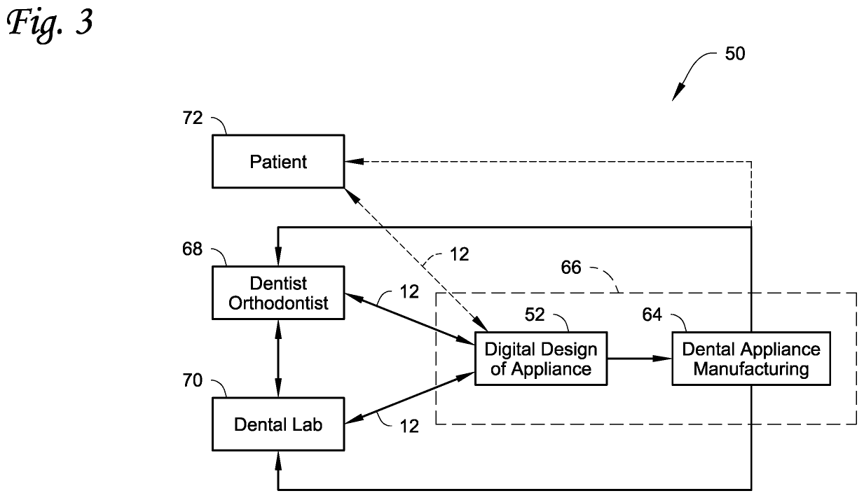

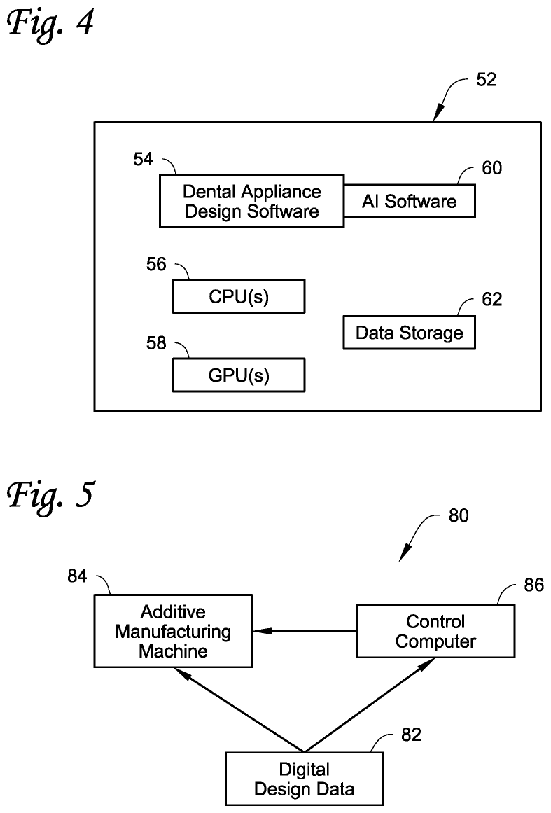

Referring to FIGS. 3 and 4, an example of a system 50 for implementing some or all of the method 10 is illustrated. The system 50 includes one or more servers 52 having stored thereon dental appliance design software 54 that is executable by one or more processors, such as CPUs 56 and/or GPUs 58. The design software 54 is configured to digitally design dental appliances. Dental appliance design software is well known in the art. One example of dental appliance design software is Appliance Designer™ Software available from Great Lakes Dental Technologies of Tonawanda, N.Y. The design software 54 can be connected to or incorporate therein artificial intelligence (AI) software 60. The design software 54, together with the AI software 60, permits the design software 54 to automatically start and optionally finish the digital design of a requested dental appliance after receipt of an order without human intervention. The server 52 can also include data storage 62 for storing the digital design during and/or after the digital design creation process.

The system 50 can also include a system 64 that manufactures the dental appliance. The system 64 is in suitable wired or wireless communication with the server 52 to receive the completed digital design from the server 52 and use the digital design to manufacture the dental appliance. The server 52 and the system 64 may be remote from one another or they may be located together, for example in a common building, as indicated by dashed box 66. The server 52 and the system 64 may be owned and controlled by separate entities, or the server 52 and the system 64 may be owned and controlled by the same entity.

As shown in FIG. 3, the server 52 can receive the order 12 directly from a dentist/orthodontist 68 or from a dental lab 70 which works with the dentist/orthodontist 68. In some embodiments, the order 12 may also be received directly from a patient 72. In some embodiments, after receipt of the order 12 and during creation of the digital design, communications can be sent from the server 52 to the dentist/orthodontist 68, to the dental lab 70 and/or to the patient 72 seeking clarification of the order 12 and asking questions. After the dental appliance is manufactured by the system 64, the dental appliance can be sent to the dentist/orthodontist 68, to the dental lab 70 which forwards the appliance to the dentist/orthodontist 68, or directly sent to the patient 72.

FIG. 5 illustrates an example of an additive manufacturing system 80 that can be used to manufacture the patient specific dental appliance from the created digital design 82. The system 80 includes an additive manufacturing machine 84 and optionally a control computer 86 that is connected to and controls the additive manufacturing machine 84 using suitable production software known in the art. The digital design 82 can be transmitted directly to the additive manufacturing machine 84 if the machine 84 includes integral data processing and control capability, or the digital design 82 can be transmitted directly to the control computer 86. In some embodiments, the digital design software used to create the digital design 82 and the production software of the control computer 86 can be integrated together, for example on the control computer 86.

In another embodiment, the patient specific dental appliance can be generated using a lost-wax casting (or investment casting) process. The digital design 82 can be used to 3D print or otherwise generate a model of the appliance from a material such as plastic, ceramic or other material. The model is then used to form a mold that can be used to form the final patient specific dental appliance. The lost-wax casting (or investment casting) process is well-known and further details of the process are not necessary.

The dental appliances can be any dental appliances that are worn by patients during a course of dental/orthodontic treatment including, but not limited to, space maintainers, palatal expanders, distalizing appliances, retainers, habit appliances, dental bands, wires, partial frameworks including, but limited to, Maryland bridges and crowns, and other dental appliances. FIGS. 6-8 illustrate non-limiting examples of digital designs of patient specific dental appliances that can be created.

FIG. 6 illustrates a digital design of a unilateral space maintainer 90 in place on a digital model of a portion of the patient's teeth. In this example, the digital design of the space maintainer 90 includes a dental band 92 and a wire 94 extending from the dental band 92. The general construction and function of a dental band and a wire in a unilateral space maintainer is known in the art. An example of a specific construction of the dental band 92 and the wire 94 can be found in U.S. Provisional Patent Application Ser. No. 63/117,120, filed on Nov. 23, 2020 and titled Patient Specific Dental Bands For Dental Appliances, and U.S. patent application Ser. No. 17/461,485, filed on Aug. 30, 2021 and titled Patient Specific Dental Appliances, each of which is incorporated herein by reference in its entirety.

FIG. 7 illustrates a digital design of a bilateral space maintainer 100 in place on a digital model of a portion of the patient's teeth. In this example, the digital design of the space maintainer 100 includes a pair of dental bands 102 and a wire 104 extending from and between the dental bands 102. The general construction and function of dental bands and a wire in a bilateral space maintainer is known in the art. An example of a specific construction of the dental bands 102 and the wire 104 can be found in U.S. Provisional Patent Application Ser. No. 63/117,120, filed on Nov. 23, 2020 and titled Patient Specific Dental Bands For Dental Appliances and U.S. patent application Ser. No. 17/461,485, filed on Aug. 30, 2021 and titled Patient Specific Dental Appliances, each of which is incorporated herein by reference in its entirety.

FIG. 8 illustrates a digital design of a palatal expander 110 in place on a digital model of a portion of the patient's teeth. In this example, the palatal expander 110 includes a pair of dental bands 112 and partial dental bands 114, and wires 116 extending from the dental bands 112, 114, and a mechanical expansion device 118, such as a screw, that spreads apart the two halves of the expander 110. The general construction and function of palatal expanders is known in the art. An example of a specific construction of the dental bands 112, 114 can be found in U.S. Provisional Patent Application Ser. No. 63/117,120, filed on Nov. 23, 2020 and titled Patient Specific Dental Bands For Dental Appliances, the entire contents of which are incorporated herein by reference.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A process of creating a dental appliance, comprising:
digitally receiving an order for the dental appliance to be created, the order including data on a patient's teeth and an identification of the dental appliance to be created;
automatically creating a portion of a digital design of the dental appliance using data from the order without initial human interaction;
thereafter continuing creation of the digital design of the dental appliance using human interaction;
and storing the digital design in data storage.

2. The process of claim 1, wherein continuing creation of the digital design of the dental appliance using human interaction comprises completing creation of the digital design of the dental appliance using the human interaction.

3. The process of claim 1, further comprising creating the dental appliance based on the digital design.

4. The process of claim 3, comprising sending the digital design to an additive manufacturing system, and creating the dental appliance using the additive manufacturing system.

5. The process of claim 3, comprising creating the dental appliance from at least one metal material.

6. The process of claim 1, wherein the dental appliance comprises a space maintainer, a palatal expander, a distalizing appliance, a retainer, or a habit appliance.

7. The process of claim 1, wherein the dental appliance comprises at least one dental band and a wire.

8. A dental appliance created using the process of claim 1.

9. An additive manufacturing system that produces a dental appliance, comprising:
an additive manufacturing machine for creating the dental appliance;
a control computer connected to and controlling the additive manufacturing machine;
a system in communication with the control computer or with the additive manufacturing machine, the system provides a digital design of the dental appliance to the control computer or to the additive manufacturing machine, wherein the system automatically creates a portion of the digital design without initial human interaction and creates a portion of the digital design using human interaction.

10. The additive manufacturing system of claim 9, wherein the additive manufacturing machine is configured to create the dental appliance from at least one metal material.

11. The additive manufacturing system of claim 9, wherein the dental appliance comprises a space maintainer, a palatal expander, a distalizing appliance, a retainer, or a habit appliance.

12. The additive manufacturing system of claim 9, wherein the dental appliance comprises at least one dental band and a wire.
